SEQUENCE LISTING (1 sequence)

SEQ ID NO: 1 - DNA, 45 bp, P. SHIGELLOIDES
dttgaatgcgaatgcaaataattttcatttgatctcaggtgtcat

(19) United States
(12) Patent Application Publication
(10) Pub. No.: US 2007/0166792 A1
(43) Pub. Date: Jul. 19, 2007

(54) Increasing hemoglobin and other heme protein production in bacteria by co-expression of heme transport genes

(76) Inventors: John S. Olson, Houston, TX (US); Douglas Henderson, Odessa, TX (US)

Correspondence Address: BAKER BOTTS L.L.P.;PATENT DEPARTMENT, 98 SAN JACINTO BLVD., SUITE 1500, AUSTIN, TX, 78701-4039, US

(21) Appl. No.: 11/724,006

(22) Filed: Mar. 14, 2007

Related U.S. Application Data
(63) Continuation of application No. PCT/US05/33027, filed on Sep. 15, 2005.
(60) Provisional application No. 60/610,108, filed on Sep. 15, 2004.
(60) Provisional application No. 60/610,109, filed on Sep. 15, 2004.
(60) Provisional application No. 60/610,110, filed on Sep. 15, 2004.

(51) Int. Cl. C12P 21/06; C12N 1/21; C07K 14/805; C12N 15/74

(52) U.S. Cl. 435/69.1; 435/471; 435/252.33; 435/252.3; 530/385

(57) ABSTRACT

The present disclosure relates to methods for increasing heme uptake in a Gram-negative bacterium comprising expressing at least one transgenic heme transport gene in the bacterium. The present disclosure also relates to heme protein production cells comprising at least one transgenic heme transport gene and a heme protein gene and plasmids comprising one or more heme transport genes and a promoter operable to promote expression of the genes by iron depletion or by the addition of an inducer molecule. The present disclosure also relates to systems for heme protein production.

### RELATED APPLICATION

This application is a continuation of International Patent Application No. PCT/US2005/033027, filed Sep. 15, 2005, which claims priority to U.S. Provisional Patent Application No. 60/610,108, filed on Sep. 15, 2004, U.S. Provisional Patent Application No. 60/610,109, filed on Sep. 15, 2004, and U.S. Provisional Patent Application No. 60/610,110, filed on Sep. 15, 2004, the full disclosures of which are incorporated herein by reference.

### TECHNICAL FIELD

The present disclosure relates to compositions and/or methods of producing compositions that include a form of hemoglobin.

### BACKGROUND

Hemoglobin (Hb) is responsible for carrying and delivering oxygen to tissues and organs in animals and has been used in development of an effective and safe oxygen carrier as an alternative to blood transfusion. Hb can be obtained easily in large quantities from bovine sources, or can be produced transgenically, so the raw material is not limiting. Such forms of Hb, however, may have numerous serious side effects when transfused into a human patient. For example, raw Hb may cause vasoconstriction, abdominal pain, and acute kidney failure. In addition, products may cause elevation of blood pressure and other problems associated with interference with smooth muscle regulation.

Some of these effects may stem from the toxicity of Hb when it is outside of a red blood cell (erythrocyte). In addition, Hb outside of a red blood cell is rapidly broken down from its tetrameric form into dimers and monomers. These products may be taken up by the kidney and impair nephrological functions.

When hemoglobin or other globins are expressed in E. coli, the heme prosthetic group must be added to the apoprotein. If this does not occur rapidly, the partially folded apoglobin is degraded by bacterial proteases. Because most strains of E. coli have native heme biosynthetic pathways, the heme prosthetic group can be provided by the bacterium. However, bacteria cannot make sufficient amounts of heme to supply the heme prosthetic group to the pool of apoglobin being generated when globins are over expressed. Moreover, laboratory strains of E. coli generally lack their own heme transport systems and thus cannot move exogenously added heme into the cell.

### SUMMARY

Accordingly, expression of hemoglobin or other globins in E. coli may be problematic. Excess hemin may get “stuck” in the outer membrane of E. coli (hemin refers to the Fe⁺ oxidation product of heme and may be used herein interchangeably). And excess hemin may be degraded to metal-free porphyrin, which may be taken up by apoHb to form photosensitive contaminants that cause degradation. Accordingly, the production of rHb is limited by the stability of the apoglobin and the ability of cells to take up heme, which is readily available from safe commercial sources. So, in order to produce therapeutic hemoglobin, a number of technical problems must be overcome, one of which is producing hemoglobin in sufficient amounts to be economically viable for use.

Therefore, there is a need for compositions, systems, and methods for producing hemoglobin that increase the uptake of heme by cells so that heme may be incorporated into the apoprotein. Increasing the uptake of heme by cells may facilitate commercial production of rHb, other important heme proteins, and/or provide additional benefits.

The compositions, systems, and methods of the present disclosure, according to certain example embodiments, may be useful for producing hemoglobin for therapeutic applications. For example, the heme utilization and transport genes from Gram-negative bacteria heme utilization systems may be co-expressed with hemoglobin genes to increase the production of intact, functional rHB and/or other heme proteins. The rHb product from this heme transport and hemoglobin gene co-expression system may, for example, be used as the starting material for a blood substitute. The other heme proteins may be used for a variety of other research, industrial, and/or pharmaceutical products.

The present disclosure, according to one specific example embodiment, relates to methods for producing hemoglobin in bacteria, and more particularly to co-expression expression of heme transport genes with human α or β globin genes, including derivatives and mutants of these genes, and with other heme proteins (including other hemoglobins, myoglobins, flavohemoglobins, peroxidases, cytochromes, cytochrome P450s, nitric oxide synthases, guanylyl cyclases, and the like).

Some embodiments of the present disclosure provide compositions comprising bacterial production cells having heme transport genes and/or human α or β globin genes, including derivatives and mutants of these genes, and any other heme protein genes. An E. coli strain may then be grown in media supplemented with heme, such that the heme transported into the cell, as well as any heme synthesized by the cell, may be available for incorporation into apohemoglobin, among other things, resulting in the production of larger quantities of stable holohemoglobin.

According to one example embodiment, the present disclosure may provide methods for increasing heme uptake in a Gram-negative bacterium comprising expressing at least one transgenic heme transport gene in the bacterium.

According to another example embodiment, the present disclosure may provide heme protein production cells comprising at least one transgenic heme transport gene and/or a heme protein gene.

According to another example embodiment, the present disclosure may provide systems for heme protein production comprising a plurality of production cells, the production cells in a growth media supplemented with heme; a first nucleic acid capable of being expressed in the production cells, the first nucleic acid may encode at least one heme protein; and a second nucleic acid capable of being expressed in the production cells, the second nucleic acid may encode at least one transgenic heme transport gene; wherein the first nucleic acid and second nucleic acid may be co-expressed in the production cells.

According to another example embodiment, the present disclosure may provide plasmids comprising one or more heme transport genes; and a promoter operable to promote expression of the genes, for example, by iron depletion or by the addition of an inducer molecule, etc.,

### BRIEF DESCRIPTION OF THE DRAWINGS

The present disclosure may be better understood through reference to the following detailed description, taken in conjunction with the following figures in which:

FIG. 1 illustrates a scheme for hemoglobin assembly in both E. coli, other microorgansims, and erythroid cells.

FIG. 2 illustrates a scheme for heme transport in P. shigelloides and related pathogens.

FIG. 3 illustrates a map of the P. shigelloides genes, Fur box, and plasmids used for the co-expression experiments with rHb0.0.

FIG. 4 illustrates measurement of holo-rHb production in the presence of added heme and with and without induction of P. shigelloides heme transport genes.

FIG. 5 illustrates E. coli BL21 (D3) cells that were co-transformed with pHUG 21.1/prHb0.0 plasmids and maintained on agar plates containing tetracycline and chloramphenicol.

FIG. 6 illustrates measurement of holo-rHb0.0 and holo-rHb(α(wt)/β(Gl6A) production in E. coli BL21 cells co-transformed with pHUG21.1.

### DETAILED DESCRIPTION

The present disclosure, according to one specific example embodiment, relates to methods for producing hemoglobin in bacteria. Such methods may be capable of producing rHb that may be harvested in quantities sufficient for use in therapeutic applications. The hemoglobin produced using such methods may be stable in cell free form, for example, it does not need to be enclosed in a red blood cell in order to remain viable when administered to a patient.

In another specific example embodiments, the present disclosure may provide co-expression of one or more heme transport genes with human α and/or β globin genes. Such co-expression may produce a starting material for a blood substitute.

In other specific example embodiments, the present disclosure may provide co-expression of one or more heme transport genes, for example, the heme utilization genes (hug), with other heme protein genes to produce large amounts of these proteins for research, industrial, and/or pharmaceutical uses.

In other specific example embodiments, the present disclosure may provide methods for producing heme proteins by transfecting bacteria with a plasmid containing heme transport genes and then inducing the expression of the corresponding transport proteins to allow the bacteria to take up added external heme and, for example, incorporate it into heme proteins, for example, hemoglobin subunits, that may be expressed in the same bacteria. This process may enhance production of native-like hemoglobin and/or other heme proteins, while reducing any contaminating metal-free porphyrins. When E. coli is the bacteria, this co-expression method may enhance heme protein production in E. coli, for example, by a factor of about two to about three, which may be within the range necessary for commercial production.

In other specific example embodiments, the present disclosure relates to bacterial production cells capable of forming rHb, and/or other heme proteins, which may co-express at least one transgenic heme transport gene with a heme protein gene such that heme uptake by the cell, or its availability to apo-rHb and/or other apoproteins, is increased.

Other example embodiments may relate to vectors that may encode at least one heme transport gene. Still other example embodiments relate to systems including E. coli, or other Gram-negative cells, expressing at least one transport gene for increased rHb or other heme protein production. Other embodiments of the disclosure relate to methods of making the above cells and/or vectors, as well as to methods of identifying promising heme transport genes, for example, from Gram-negative bacteria.

Certain example embodiments of the present disclosure may be used in conjunction with existing rHb and/or other heme protein technologies. For example, it may be used in connection with two co-filed applications having U.S. Provisional Application Ser. Nos. 60/610,110 and 60/610,108, as well as U.S. Pat. Nos. 6,455,676; U.S. Pat. No. 6,204,009; U.S. Pat. No. 6,114,505; U.S. Pat. No. 6,022,849; and U.S. Patent Publication No. 2003 0017537.

Higher levels of hemoglobin production may be achieved in bacteria by enhancing the rate of uptake of externally added heme. Accordingly, one example embodiment of the present disclosure may be a method to enhance uptake includes incorporating the heme transport system from a Gram-negative bacteria, such as P. shigelloides, into another bacterium, such as E. coli.

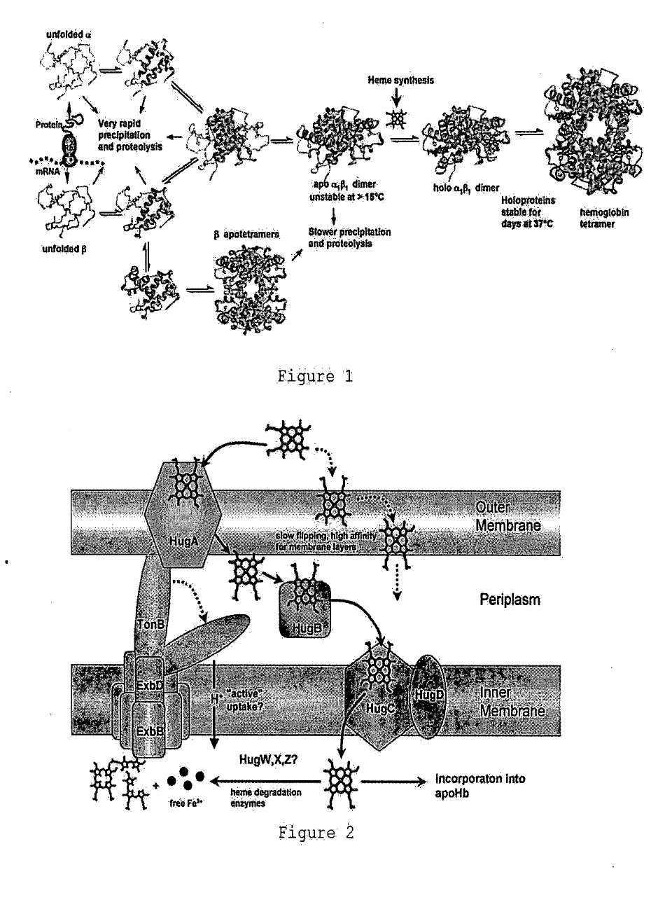

The assembly of hemoglobin in either bacteria or in animal erythroid cells (FIG. 1) may involve ribosomal synthesis of two different protein chains or subunits (α, 141 amino acids and β, 146 amino acids). Without being limited to any particular mechanism of action or theory, newly synthesized α and β subunits do not appear to have any well-formed structure in the absence of a partner, and α and β subunits first assemble to form an α₁β₁ dimer, which is also unstable (apo α₁β₁ dimer in FIG. 1, in which the suffix apo means no heme is bound and the protein has no “red” color). Only after heme (iron containing red pigment) is bound is the protein stabilized and resistant to degradation. Thus, hemoglobin synthesis in bacteria may be limited by the availability of heme. Newly formed α and β proteins that are unable to find heme may tend to precipitate or be degraded by bacterial enzymes.

Without being limited to any particular mechanism of action or theory, heme synthesis by E. coli may be too slow to keep up with induced synthesis of recombinant hemoglobin from high copy number plasmids, and thus little hemoglobin is made in the absence of added heme. Heme uptake by conventional laboratory strains of E. coli is generally inefficient, requiring careful selection of the bacterial strain (usually based on JM109 cells) and the addition of a large excess of external heme. Most of the added heme remains in the cell wall of gram-negative bacteria, where it is not accessible to hemoglobin. Some of the added heme is also degraded to metal-free porphyrin and then taken up by apo-rHb to form photosensitive contaminants that can degrade stored samples. Similar problems occur for the expression of other heme proteins in E. coli and other bacteria, including the expression of other hemoglobins, myoglobins, peroxidases, cytochromes, cytochrome P450s, nitric oxide synthases, guanylyl cyclases, etc.

The present disclosure, according to certain example embodiments, may lower the rate of apo-rHb degradation and facilitate efficient heme uptake by co-expressing hemoglobin or other heme proteins with a set of heme transport proteins that may facilitate the uptake of external heme for incorporation into the newly synthesized globins and/or other apo-heme proteins. In specific example embodiments, both the rate of heme uptake and the extent of holoprotein production may be increased by co-expressing the heme transport genes of a Gram-negative baterium, with the rHb genes in a bacterial cell, such as E. coli. This strategy may also be adapted and applied to production of all heme proteins in E. coli and other Gram-negative bacteria, including other hemoglobins, myoglobins, flavohemoglobins, peroxidases, cytochromes, cytochrome P450s, nitric oxide synthases, and guanylyl cyclases, among others. Thus, some example embodiments of the present disclosure may facilitate hemin uptake and holoHb production and, at the same time, reduce or eliminate the incorporation of modified and/or metal free porphyrins by adding only the amount of heme needed for incorporation into newly synthesized apoglobin chains.

Heme transport and rHb production may be optimized in bacterial systems (e.g., E. coli) by: (i) choosing the appropriate bacterial production cell that can utilize express the heme transport genes and over-express holo-rHB or other heme proteins; (ii) choosing the appropriate bacterial heme transport genes for incorporation into the bacterial production cell such that the heme transport is efficient; (iii) modifying and/or replacing the Fur transcription factor in P. shigelloides hug heme transport gene systems so induction can be carried out under high iron conditions; (iv) constructing a single vector containing the rHb or other heme protein genes under control of the T7 or tac promoter and heme transport genes (as well as other helper genes such as AHSP and/or methionine amino peptidase) under control of an alternative promoter/inducer system to allow lower levels of expression; and (v) incorporating the heme transport genes into the bacterial production cell's chromosome. Thus, for example, heme transport system genes may be on the same plasmid as the hemoglobin genes, or they may be in the chromosome of E. coli containing a plasmid that encodes hemoglobin genes. Some or all of these steps, such as those relating to heme transport genes, may be truncated or omitted as needed for a given rHb or heme protein system design. Systems may be evaluated using rHb expression assays, including both CO derivative and Zn-column assays.

Bacterial production strain. As discussed above, the cell containing one or more heme transport genes may be a strain of E. coli, such as BL21. Other suitable E. coli strains include, but are not limited to, laboratory strains of E. coli, HB101, SGE1661, DH5α, JM109, 1017 (a sideropore defective strain of E. coli 101), E. coli protease mutants (e.g., Lon and C1pP mutants), and DHE-1 (a hemA mutant). Other suitable bacterial strains include, but are not limited to, nonpathogenic strains of S. dysenteriae.

Heme transport genes. A number of bacteria, typically pathogens, have heme transport systems (Genco, CA et al. (2001) Mol Micro 39:1-11). Bacteria use these systems to acquire heme as an iron source. Most bacterial pathogens cannot survive in the host without a means of acquiring iron (Braun, V et al. (2002) FEBS Letters 529:78-85). Stojiljkovic and Perkins-Baldwin have presented an extensive review of the heme processing genes found in both Gram-negative and Gram-positive organisms (Stojiljkovic, I. et al. (2002) DNA Cell Biol 21, 281-295). While iron is plentiful in the host, most is biologically unavailable unless the pathogen has a special high affinity iron transport system, such as a heme iron transport system.

Several bacterial heme transport systems can be reconstituted into laboratory strains of E. coli, which lack heme transport systems, allowing the organism to transport heme into the cell(Daskaleros, PA et al. (1991) Infect Immun 59:2706-2711; Stojiljkovic, I et al. (1992) EMBO 11:4359:4367; Henderson, DP et al. (1993) Mol Micro 7:461-469; Mills, M et al. (1995) J Bacteriol 177:3004-3009; Torres, AG et al. (1997) Mol Micro. 23:825-833.; Ghigo, JM et al. (1997)J Bacteriol 179:3572-3579). Accordingly, heme transport genes suitable for use in the present disclosure may be derived from a Gram-negative bacterium having a heme transport system including, but are not limited to, Plesiomonas shigelloides, Shigella dysenteriae, Vibrio cholerae, Yersinia enterocolitica, Yersinia pestis, E. coli 0157:H7, and Serratia marcescens.

In one aspect, the heme transport gene may be derived from S. dystenteriae shuA , the heme receptor gene (Mills, M et al. (1995) J Bacteriol 177:3004-3009). Because E. coli and S. dysenteriae are closely related, the E. coli TonB system functions with ShuA. Heme transport occurs in E. coli 1017, with only shuA present. In another aspect, the heme transport genes may be formed using the entire heme iron utilization system from S. dystenteriae with the following genes: shuA, the heme receptor gene; shuT, the periplasmic permease gene; shuW, X, and Y whose functions are not clear; shuU and V, the inner membrane permease genes; and shuS, which encodes a protein thought to be involved in heme iron utilization but not heme transport. In another aspect, the heme transport genes may be derived from S. dystenteriae using the entire heme iron utilization system as described above, but with shuS interrupted with a chloramphenicol cassette.

In another aspect, the heme transport gene may be derived from Y. enterocolitica using the wild type hemR gene, which may be sufficient to allow heme transport in a heme biosynthesis mutant of E. coli. In another aspect, the heme transport gene may be formed from the hemR gene with a mutation that converts the histidine 192 in HemR to a threonine. This mutation, when placed in a heme biosynthesis mutant of E. coli, permitted slightly better growth in a heme transport plate assay than did a plasmid containing the wild type hemR gene.

In another aspect, the heme transport gene may be derived from S. marcescens using the heme receptor gene, hasR. HasR functions with the E. coli TonB system and allows heme transport (Ghigo, JM et al. (1997)J Bacteriol 179:3572-3579).

Heme transport gene promoters. In some instances, heme transport genes may be expressed most efficiently during iron starvation. But these conditions may not be conducive to the maintenance of viable cells that are capable of overproducing globin proteins. In this regard, the promoters of the heme transport genes may be modified to increase the expression of the genes, for example, so the genes are expressed at a more constitutive level, which may allow for increased heme transport in iron replete media.

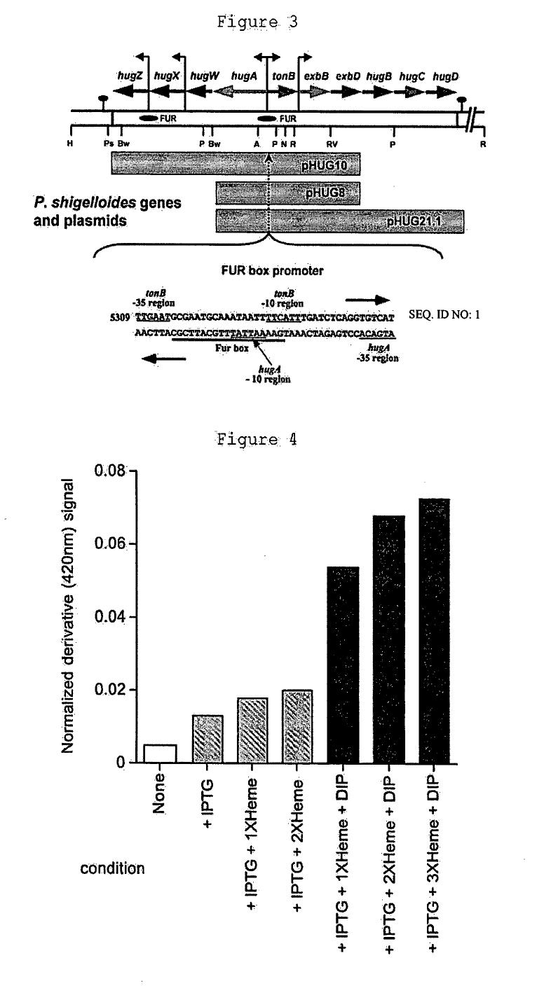

In the case of heme transport systems regulated, at least in part, by the Fur repressor protein (e.g., expressed under low iron conditions and not expressed well under high iron conditions) (Griggs, D W et al. (1989) J Bacteriol 171:1048-1054; for review, see Andrews, S C et al. (2003) FEMS Microbiol Rev 27:215-237). For example, in P. shigelloides, a putative Fur box (the sequence in the promoter to which Fur binds) was identified that overlaps the putative divergent overlapping promoters for hugA and tonB (See FIG. 3). Both hugA and tonB were shown to be iron regulated by placing the promoter in both orientations in front of a promoterless lacZ gene.

Accordingly, in some example embodiments, the Fur box may be modified. The Fur box may be modified using methods known in the art. For example, highly conserved sequences in the Fur box may be altered by PCR mediated site direcged mutagenesis (Sambrook, J. and D W Russell. 2001. Molecular Cloning: A Laboratory Manual. Cold Spring Harbor Laboratory Press. Cold Spring Harbor, N.Y.). Such modifications may be chosen to moderately increase expression of heme transport system genes (e.g., hugA and tonb). But high expression may not be desired because, among other things, over expression of an outer membrane receptor gene or tonb may be harmful to the cell. This may be especially relevant when hemoglobin genes are also over expressed at the same time in the strain.

Vectors. The heme transport genes, heme protein genes, and/or human α or β globin genes may be on the same or different plasmids for expression and/or co-expression in the production cell. Such plasmids may be constructed using recombinant DNA techniques known in the art.

For example, according to one example embodiment, the heme transport genes may be on one plasmid and the heme protein genes on another plasmid. In another example embodiment, the heme transport genes and the heme protein genes may be present on the same plasmid. In another example embodiment, only the human α or β globin genes or heme protein genes are present on a plasmid. In this embodiment the E. coli strain may comprise the heme transport genes from a Gram-negative bacterial heme transport system incorporated into the E. coli chromosome. Such E. coli strains may be constructed, for example, by marker exchange or by transposon mutagenesis. Once constructed, the E. coli strain may then be transformed with the plasmid.

The following discussion relates to specific example embodiments of the present disclosure.

As discussed above, one example of a heme transport system may be derived from P. shigelloides heme utilization genes (hug). A schematic diagram of the P. shigelloides system is shown in FIG. 2. In FIG. 2, the structural interpretation of the tonB system was taken from Postle, K. et al. (2003) Mol Microbiol 49, 869-882; Seliger, S. S. et al. (2001) Mol Microbiol 39, 801-812 and the overall system from Stojiljkovic, I. et al. (2002) DNA Cell Biol 21, 281-295. Although hemin incorporation into the outer layers of phospholipids membranes is fast, non-facilitated flipping of the heme propionates is very slow (Light, W. R., 3rd et al. (1990) J Biol Chem 265, 15623-15631; Light, W. R., 3rd et al. (1990) J Biol Chem 265, 15632-15637).

The hug genes have been cloned and genetically characterized by expressing plasmids containing different sets of the hug genes and then measuring the extent of heme transport in the transfected E. coli (Henderson, D. P. et al. (2001) J Bacteriol 183, 2715-2723). These proteins correspond in function and sequence to the heme transport genes found in V. cholerae and related Vibrio species (Henderson, D. P. et al. (1994) J Bacteriol 176, 3269-3277; Henderson, D. P. et al. (1994) Infect Immun 62, 5120-5125; Henderson, D. P. et al. (1993) Mol Microbiol 7, 461-469) and other gram negative pathogens (Stojiljkovic, I. et al. (2002) DNA Cell Biol 21, 281-295).

As discussed above, expression of heme transport genes in P. shigelloides is controlled by the Fur transcription factor, which attaches to its corresponding promoter region when iron is bound (Bagg, A. et al. (1987) Biochemistry 26, 5471-5477; Litwin, C. M. et al. (1993) Clin Microbiol Rev 6, 137-149; Pohl, E. et al. (2003) Mol Microbiol 47, 903-915). Iron depletion causes dissociation of the Fur repressor protein and turns on transcription of the hug system.

Heme uptake is driven by the TonB energy transduction complex that utilizes proton gradients to drive active transport of iron siderophores, vitamin B12, and heme across the lipophilic outer cell wall and into the periplasm of most gram negative bacteria (Postle, K. et al. (2003) Mol Microbiol 49, 869-882; Letoffe, S. et al. (2004) J Bacteriol 186, 4067-4074). Heme is then bound by the soluble HugB binding protein and transported across the cell membrane by HugC and HugD. Little is known about the latter process.

The hugx, Y, and Z genes are involved in heme degradation and iron release and are not required for uptake. These degradation genes are generally excluded from co-expression vectors in most example embodiments of the present disclosure. Additionally, in many example embodiments, it may be preferable to use the minimum number of heme transport and tonb or equivalent genes required for efficient heme transport and enhanced rHb production in E. coli (see FIG. 3).

Thus, in one example embodiment of the present disclosure, the entire P. shigelloides heme transport gene region, in which the genes are contiguous, was subcloned into the low copy number plasmid pACYC184 (New England BioLabs). The heme degradation genes, hugw, hugx, and hugz, were then cleaved to create plasmids containing only the outer membrane HugA heme receptor, the TonB/ExbB/ExbD energy transduction systems, and the transport genes, hugb, hugC, and hugD (FIG. 3). hug genes have been co-expressed with the genes for wild-type sperm whale myoglobin (pWTMb) and poorly expressing mutants. Significant observations from these experiments are: (a) production of wild-type and unstable mutant rMb can be enhanced by co-expression with the pHUG8 vector (FIG. 3); (b) no enhancement with pHUG10 occurs, presumably because HugW, HugX, HugZ degrade added heme; and (c) E. coli BL21 colonies could not be maintained when co-transformed with the pHUG21.1 and pWTMb plasmids. The latter problem may be related to the markedly high affinity of myoglobin for heme, which can cause cytoplasmic iron depletion and prevent aerobic growth. In contrast, there is no problem maintaining BL21 cells containing pHUG21.1 and prHb0.0.

Initial rHb production experiments using hemoglobin with the prHb0.0/pHUG21.1 system in E. coli BL21 have been conducted (see FIGS. 3-5). rHb expression was induced with IPTG in the presence of varying amounts of heme and in the presence and absence of 2,2-dipyridine (DIP, 62.5 μM). DIP causes iron depletion, which de-represses the Fur promoter, leading to induction of the hug genes. The results of one initial experiment are shown in FIG. 4. There is a small amount of rHb production after induction with IPTG that increases slightly with heme addition. In this experiment induction of the hug genes by DIP causes a dramatic 3 to 4-fold increase in rHb production in the presence of heme and increases as more is added. This result strongly supports the premise that the rate of heme transport is limiting rHb production and indicates that embodiments of the present disclosure using the pHUG21.1 co-expression system, as well as other embodiments, enhance rHb expression sufficiently for large-scale production.

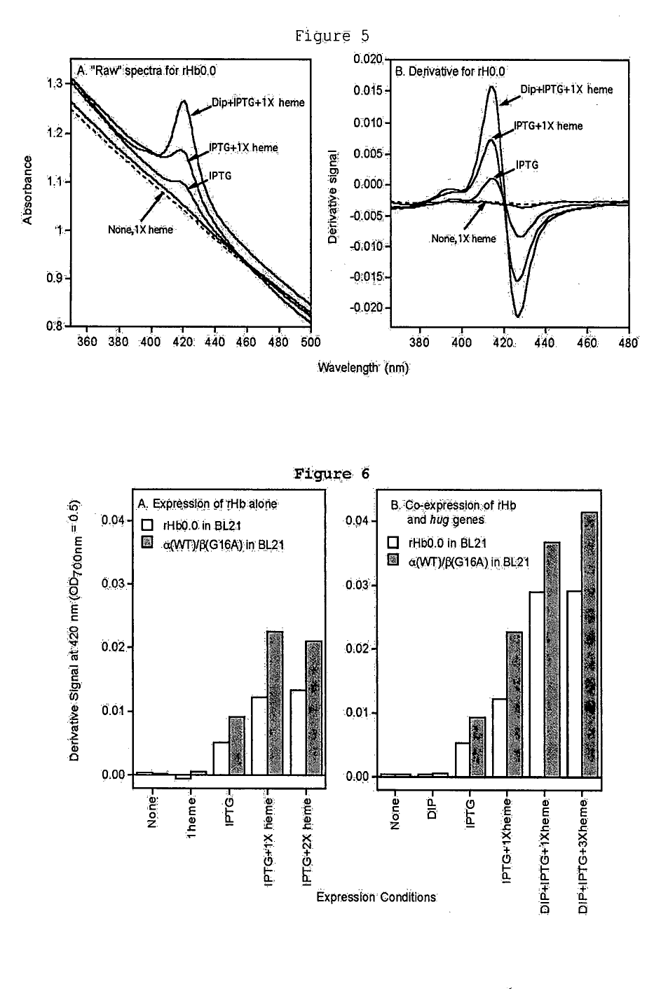

A second set of data for rHb production in E. coli are shown in greater detail in FIG. 5 and emphasize the dramatic effect of co-expression of the hugA, hugb, hugc, hugo, exbB, exbD, and tonB genes from P. shigelloides on rHb production (see also FIGS. 2-4). These data were collected and processed digitally at Rice University; those in FIG. 4 were collected at the University of Texas, Permian Basin. Briefly, tubes containing 5 ml of LB broth were inoculated and then grown overnight at 37° C. Various additions were made to the cultures including IPTG, heme (increments of 10 μM total=1X), and Dip (63 μM total), and the cultures were incubated at 37° C. for another 16 hours. Then the cells were pelleted, resuspended to 0.5 absorbance units at 700 nm in Tris buffer, pH 7.5, and equilibrated with 1 atm of CO for 15 minutes to ensure HbCO formation and no further cell growth. Spectra of these samples were recorded as shown in FIG. 5A. Dithionite was added to some samples, but no differences in the 420 nm peak heights were seen. The first derivatives of the observed spectra are shown in FIG. 5B. No rHbCO was detected in the absence of IPTG induction, regardless of whether heme or Dip was added to the cultures.

In these data, expression of rHb was induced with IPTG and the P. shillegoides hug genes were induced with DIP, which chelates iron, causing release of the Fur repressor from the pHUG21.1 plasmid. The enhancement of hemoglobin production in these experiments was dramatic and visible by eye as red bacterial pellets. The effect was clearly seen in the absolute absorbance spectrum and easily quantified in the derivative spectrum by the peak to trough height (FIG. 5). The data shown in FIG. 5 confirm the results presented in FIG. 4.

Mutant apohemoglobin α(wt)/β(G16A) is much more resistant to GdmC1-induced denaturation than the wild-type rHb0.0 apoprotein. The mutation occurs at the A13 helical position and extends this initial helix by one amino acid residue. A comparison of the holoprotein yields of wild-type and α(wt)/β(G16A) rHb in small cultures in the absence and presence of heme and hug genes is shown in FIG. 6. FIG. 6 shows that enhancing apohemoglobin stability also increases holoprotein expression levels. In the absence of expression of the P. shigelloides hug genes, the mutant rHb expression level was roughly twice that of the wild-type protein (columns with no DIP in FIG. 6). This ratio became smaller when heme transport efficiency was increased by the hug transport system, but in all cases, more intact mutant protein was made. Thus, the heme transport aspects of the present disclosure may be usefully combined with other methods of increasing hemoglobin production, such as mutation of α or hemoglobin to form more stable apo-rHb.

Briefly, the assays from FIG. 6 were performed as described in FIG. 5. The derivative signal was the peak to trough distance in the derivative spectra at 420 nm (FIG. 5B). Note that almost twice as much of the β(G16A) mutant was expressed compared to wild-type rHb0.0 This two-fold enhancement of expression occurred in the absence and presence of heme (FIG. 6A). Co-expression of the hug genes (+Dip) enhanced the production of both proteins markedly and reduced the differences in the levels between the wild-type and mutant rHbs (FIG. 6B).

Although the present disclosure and its advantages have been described in detail, it should be understood that various changes, substitutions, and alternations can be made herein, including co-expression of P. shigelloides heme transport genes and those from other bacteria with other heme proteins, without departing from the spirit and scope of the disclosure as defined by the following claims.

What is claimed is:

1. A method for increasing heme uptake in a Gram-negative bacterium, the method comprising expressing in the bacterium a nucleic acid having a transgenic heme transport gene.

2. A method according to claim 1, further comprising co-expressing a nucleic acid having a heme protein gene.

3. A method according to claim 1, wherein the nucleic acid having a heme protein gene encodes a transgenic α globin gene, a transgenic β globin gene, or both a transgenic α globin gene and a transgenic β globin gene.

4. A method according to claim 1, further comprising co-expressing a nucleic acid encoding a recombinant hemoglobin subunit or other heme protein.

5. The method according to claim 1, wherein the Gram-negative bacteria is Escherichia coli or a nonpathogenic strain of Shigella dysenteriae.

6. The method according to claim 1, wherein the transgenic heme transport gene is derived from a bacterium selected from the group consisting of Plesiomonas shigelloides, Shigella dysenteriae, Vibrio cholera, Yersinia enterocolitica, Yersinia pestis, Escherichia coli, Serratia marcescens, and combinations thereof.

7. The method according to claim 1, wherein the transgenic heme transport gene is derived from Plesiomonas shigelloides and is selected from the group of genes consisting of hugA, tonB, exbB, exbD, hugB, hugc, hugD, a derivative thereof, and combinations thereof.

8. The method according to claim 1, wherein the transgenic heme transport gene is derived from Shigella dysenteriae and is selected from the group of genes consisting of shuA, shuT, shuW, shuX, shuy, shuU, shuv, shuS, a derivative thereof, and combinations thereof.

9. The method according to claim 1, wherein the transgenic heme transport gene is derived from Yersinia enterocolitica and is selected from the group of genes consisting of hemR, a derivative of hemR, and combinations thereof.

10. The method according to claim 1, wherein the transgenic heme transport gene is derived from Serratia marcescens and is selected from the group of genes consisting of hasR, a derivative of hasR, and combinations thereof.

11. The method according to claim 1, wherein the transgenic heme transport gene is in a plasmid.

12. The method according to claim 1, wherein the transgenic heme transport gene is in the bacterium's chromosome.

13. A heme protein production cell comprising a transgenic heme transport gene and a heme protein gene.

14. A heme protein production cell according to claim 13, wherein the heme protein gene is a transgenic α globin gene, a transgenic β globin gene, or both.

15. A heme protein production cell according to claim 13, wherein the cell is a Gram-negative bacterium.

16. A heme protein production cell according to claim 13, wherein the cell is Escherichia coli or a nonpathogenic strain of Shigella dysenteriae.

17. A heme protein production cell according to claim 13, wherein the heme protein gene is a transgenic apo-heme protein gene.

18. A heme protein production cell according to claim 13, wherein the heme protein gene is a transgenic apo-heme protein gene selected from the group of genes consisting of a hemoglobin, a myoglobin, a peroxidase, a cytochrome, a cytochrome P450, a nitric oxide synthase, a guanylyl cyclase, a derivative thereof, and combinations thereof.

19. A heme protein production cell according to claim 13, wherein the transgenic heme transport gene is derived from a bacterium selected from the group consisting of Plesiomonas shigelloides, Shigella dysenteriae, Vibrio cholera, Yersinia enterocolitica, Yersinia pestis, Escherichia coli, and Serratia marcescens, and combinations thereof.

20. A heme protein production cell according to claim 13, wherein the transgenic heme transport gene is derived from Plesiomonas shigelloides and is selected from the group of genes consisting of hugA, tonB, exbB, exbD, hugB, hugc, hugD, a derivative thereof, and combinations thereof.

21. A heme protein production cell according to claim 13, wherein the transgenic heme transport gene is derived from Shigella dysenteriae and is selected from the group of genes consisting of shuA, shuT, shuW, shuX, shuy, shuU, shuV, shuS, a derivative thereof, and combinations thereof.

22. A heme protein production cell according to claim 13, wherein the transgenic heme transport gene is derived from Yersinia enterocolitica and is selected from the group of genes consisting of hemR, a derivative or hemR, and combinations thereof.

23. A heme protein production cell according to claim 13, wherein the transgenic heme transport gene is derived from Serratia marcescens and is selected from the group of genes consisting of hasR, a derivative of hasR, and combinations thereof.

24. A system for heme protein production comprising:
a plurality of production cells in a growth media supplemented with heme;
a first nucleic acid capable of being expressed in the production cells, the first nucleic acid encoding a heme protein; and
a second nucleic acid capable of being expressed in the production cells, the second nucleic acid encoding a transgenic heme transport gene;
wherein the first nucleic acid and second nucleic acid are co-expressed in the production cells.

25. A system according to claim 24, wherein the system produces a recombinant hemoglobin.

26. A system according to claim 24, wherein the system produces a heme protein selected from the group of proteins consisting of a hemoglobin, a myoglobin, a peroxidase, a cytochrome, a cytochrome P450, a nitric oxide synthase, a guanylyl cyclase, a derivative thereof, and combinations thereof.

27. A system according to claim 24, wherein the first nucleic acid is in a plasmid and the plasmid is within the production cells.

28. A system according to claim 24, wherein the first nucleic acid is in the chromosome of the production cells.

29. A plasmid comprising:
a heme transport gene; and
a promoter operably linked to the heme transport gene.

30. A plasmid according to claim 29, wherein the heme transport gene is derived from a bacterium selected from the group consisting of Plesiomonas shigelloides, Shigella dysenteriae, Vibrio cholera, Yersinia enterocolitica, Yersinia pestis, Escherichia coli, Serratia marcescens, and combinations thereof.

31. A plasmid according to claim 29, wherein the heme transport gene is selected from the group of genes consisting of an outer membrane heme receptor gene, tonB, exbB, exbD, a periplasmic heme binding protein gene, an inner membrane heme transport gene, and combinations thereof.

32. A plasmid according to claim 29, wherein the heme transport gene is derived from Plesiomonas shigelloides and is selected from the group of genes consisting of hugA, tonB, exbB, exbD, hugB, hugc, hugD, a derivative thereof, a derivative thereof, and combinations thereof.

33. A plasmid according to claim 29, wherein the heme transport gene is derived from Shigella dysenteriae and is selected from the group of genes consisting of shuA, shuT, shuW, shuX, shuy, shuU, shuV, shuS, a derivative thereof, and combinations thereof.

34. A plasmid according to claim 29, wherein the heme transport gene is derived from Yersinia enterocolitica and is selected from the group of genes consisting of hemR, a derivative of hemR, and combinations thereof.

35. A plasmid according to claim 29, wherein the heme transport gene is derived from Serratia marcescens and are selected from the group of genes consisting of hasR, a derivative of hasR, or combinations thereof.
